(19) United States
(12) Patent Application Publication
(10) Pub. No.: US 2024/0404704 A1
(43) Pub. Date: Dec. 5, 2024

(54) WEARABLE COMPANION FOR DETECTING THE CYTOKINE RELEASE SYNDROME

(71) Applicant: PFIZER INC., New York, NY (US)

(72) Inventors: Anthony LAMBROU, New York, NY (US); Ofer WAKS, New York, NY (US); Caroline J. HOANG, Peapeck, NJ (US); Ahsan HUDA, New York, NY (US); Priti RAMACHANDRAN, New York, NY (US); Timothy DOUGHERTY, Collegeville, PA (US); Dennis HANCOCK, Collegeville, PA (US); Joshua RAYSMAN, San Diego, CA (US); Solomon RAVICH, New York, NY (US); Adam FITZGERALD, Boston, MA (US)

(21) Appl. No.: 18/722,420

(22) PCT Filed: Dec. 22, 2022
(86) PCT No.: PCT/IB2022/062653

§ 371 Date: Jun. 20, 2024

Related U.S. Application Data
(60) Provisional application No. 63/293,492, filed on Dec. 23, 2021.

(51) Int. Cl. G16H 50/20; A61B 5/01; A61B 5/021; A61B 5/024; G06N 20/00; G16H 10/60; G16H 50/50

(52) U.S. Cl. CPC G16H 50/20; A61B 5/01; A61B 5/021; A61B 5/024; G06N 20/00; G16H 10/60; G16H 50/50

(57) ABSTRACT

Embodiments disclosed herein may provide digital medicine support for predicting and mitigating side effects of bispecific antibody treatment (e.g., Pfizer's Elranatamab) for myeloma patients. The side effects may include cytokine release syndrome (CRS), infection (e.g., sepsis), neurotoxicity (e.g., peripheral neuropathy), cytopenia (e.g., neutropenia), etc. These side effects may be predicted based on passive collection of data from patient wearables, patient entered data on healthcare application, and other data such as bloodwork data. Trained machine learning models may be used for predicting the side effects. As the side effects may be predicted before their onsets, a proactive intervention may be feasible to improve the healthcare outcomes for myeloma patients.

### RELATED APPLICATIONS

This application claims priority to U.S. Provisional Application No. 63/293,492 filed on Dec. 23, 2021, of which has been incorporated herein by reference in its entirety.

### FIELD

The subject matter presented herein is directed to systems and methods for monitoring myeloma treatments. Specifically, systems and methods for monitoring side effects such as cytokine release syndrome associated with the treatment of myeloma.

### BACKGROUND

Myeloma, also referred to as multiple myeloma, is a cancer of plasma cells. The cancerous plasma cells may have a rapid growth and crowd out normal cells such as red blood cells, platelets, and other white bloods cells. A form of myeloma treatment may include bispecific antibodies. A bispecific antibody may engage two different targets at once: one arm of the antibody may bind directly to specific antigens on the cancer cells and the other arm may activate and bring a patient's own T-cells from the immune system closer to kill the cancer cells. However, there may be side effects of bispecific antibody treatment: a patient may have a likelihood of developing cytokine release syndrome (CRS) generally within the first 48 hours of the bispecific antibody treatment and may also develop other conditions such as infection (e.g., sepsis), neurotoxicity (e.g., peripheral neuropathy), and cytopenia (e.g., neutropenia) post bispecific antibody treatment.

The conventional side effect management for bispecific antibody treatment is wasteful and also inadequate. For instance, conventional CRS management involves a minimum 48 hours of hospital stay after a session of bispecific antibody treatment. However, clinical trials of a bispecific antibody treatment—Pfizer's Elranatamab—has found that that only 50-70% of the patients may experience some CRS. More specifically, about 25-35% may experience grade 1 CRS, 25-35% may experience grade 2 CRS, and 30-50% do not experience any CRS at all. Only grade 2 CRS, i.e., only 25-35% of the patients, may require hospital level intervention. Conventionally, all patients are made to stay at the hospital to observe for any onset of CRS. Therefore, the hospital-stay for 65-75% of the patients, i.e., 25-35% patients developing grade 1 CRS requiring just mild interventions and 30-50% not developing any CRS, is wasteful.

Furthermore, conventional side effect management for other conditions such as infection, neurotoxicity, cytopenia, etc. is based on sporadic clinical encounters—with limited patient-clinician face time. The sporadic clinical encounters just provide an incomplete picture of the patient's condition, often based on biased information (e.g., recall bias) from the patient. Therefore, the conventional clinical encounters do not adequately detect the risks in a timely fashion. For example, myeloma patients—especially after the bispecific antibody treatment—may have weakened immune systems that may make them prone to infections such as sepsis. Infections have been found to be a major cause of death, particularly for patients with triple class refractory multiple myelomas. Early intervention may generally be effective in mitigating the infections, however, the sporadic clinical encounters may not be adequate to detect the onset of infections guide such early interventions.

Neurotoxicity and associated peripheral neuropathy may develop from other myeloma medications, e.g., proteasome inhibitors, immunomodulatory drugs (IMiDs), which are generally taken prior to the bispecific antibody treatments. As the onset of these diseases is not necessarily fast and dramatic, there may not be adequate number of clinical encounters for the patient to escalate the relatively milder symptoms to the clinicians. Even if there were multiple clinical encounters, patients may not necessarily (e.g., due to recall bias) escalate these relatively milder symptoms. Cytopenia may occur throughout the bispecific antibody treatment regime. Cytopenia imposes additional burden on the patients as it requires frequent blood testing for identification. Frequent blood testing may not typically be convenient for myeloma patients, it may require multiple visits to the lab. Sporadic clinical encounters and sporadic blood testing are inadequate for a timely detection of cytopenia.

### SUMMARY

In some embodiments, the present disclosure relates to a digital medicine companion for bispecific antibody treatments for myeloma. The present disclosure further relates to predicting the side effects associated with the bispecific antibody treatments and proactively managing the predicted side effects.

For instance, a machine learning model may be used to predict a likelihood of a patient developing CRS after a bispecific antibody treatment (e.g., Pfizer's Elranatamab). To that end, healthcare data from patients—actively with the patients entering the data on an application and/or passively with a device capturing the healthcare data—may be collected in relation to a bispecific antibody treatment. Additional data such as bloodwork data may also be collected. The collection period for the data may begin at a predetermined time before the treatment (e.g., from a few days before). Using the collected data, the machine learning model may output a likelihood of the patients developing CRS and notifications may generated for clinicians' dashboards. The notifications may provide clinical decision support whether a patient should stay at the hospital or may be discharged for at-home monitoring, based on the likelihood of developing CRS.

Additionally or alternatively, other side effects associated with bispecific antibody treatments may be predicted and proactively managed. Patients being monitored at home may actively enter healthcare data in a healthcare application (e.g., installed on a smartphone). Other devices such as wearables and invisible sensors may passively collect healthcare data. Additionally, an at home blood monitoring system (e.g., blood collection kits mailed to patients' home) may provide blood related data. A machine learning model, using these different types of collected data, may predict whether the patient may likely develop side effects such as CRS, neurotoxicity, or cytopenia. Based on the predictions, one or more alert notifications may be sent to the patient facing interfaces (e.g., to encourage the patients to contact their clinicians) and/or clinician interfaces (e.g., to encourage the clinicians to contact their patients).

In an embodiment, a computer implemented method may be provided. The method may include retrieving, prior to a treatment, a first health data entered by a patient on a prescribed application being executed by a patient computing device; retrieving, prior to the treatment, a second health data passively collected by a prescribed wearable device; deploying a machine learning model on the first health data and the second health data to determine whether the patient will likely experience cytokine release syndrome after the treatment; and in response to the determination that the patient will likely experience the cytokine release syndrome, triggering a notification on a clinician dashboard.

In another embodiment, another computer implemented method may be provided. The method may include retrieving, after a treatment, a first health data entered by a patient on a prescribed application being executed by a patient computing device; retrieving, after the treatment, a second health data passively collected by a prescribed wearable device; deploying a machine learning model on the first health data and the second health data to determine whether the patient will likely experience an adverse health condition; and in response to the determination that the patient will likely experience the adverse health condition, triggering a notification on a clinician dashboard.

In yet another embodiment, a system is provided. The system may include one or more processors; and a non-transitory storage medium storing computer program instructions that when executed by the one or more processors cause the system to perform operations comprising: retrieving a first health data entered by a patient, undergoing bispecific antibody treatment, on a prescribed application being executed by a patient computing device; retrieving a second health data passively collected by a prescribed wearable device worn by the patient; deploying a machine learning model on the first health data and the second health data to determine whether the patient will likely experience an adverse health condition; and in response to the determination that the patient will likely experience the adverse health condition, triggering one or more notifications.

### BRIEF DESCRIPTION OF DRAWINGS

Other objects and advantages of the present disclosure will become apparent to those skilled in the art upon reading the following detailed description of exemplary embodiments and appended claims, in conjunction with the accompanying drawings, in which like reference numerals have been used to designate like elements, and in which:

FIG. 1 shows a block diagram of an illustrative operating environment for employing one or more embodiments of this disclosure.

FIG. 2 shows a block diagram of an architecture of an illustrative computing device that may perform one or more functions, according to the several embodiments of this disclosure.

FIG. 3 shows a block diagram of an illustrative architecture of operating environment, wherein one or more embodiments disclosed herein may be employed.

FIG. 4 shows a flow diagram of an illustrative method of training a prediction model for CRS prediction, according to the several embodiments of this disclosure.

FIG. 5 shows a flow diagram of an illustrative method of deploying a prediction model for CRS prediction, according to the several embodiments of this disclosure.

FIG. 6 shows a flow diagram of an illustrative method of generating a model for a baseline health behavior, according to the several embodiments of this disclosure.

FIG. 7 shows a flow diagram of an illustrative method of comparing recent healthcare data with the baseline health behavior, according to several embodiments of this disclosure.

FIG. 8A shows an illustrative process flow of CRS prediction for bispecific antibody treatment, according to several embodiments of this disclosure.

FIG. 8B shows an illustrative process flow of at-home monitoring of a patient receiving a bispecific antibody treatment, according to several embodiments of this disclosure.

The figures are for purposes of illustrating example embodiments, but it is understood that the present disclosure is not limited to the arrangements and instrumentality shown in the drawings. In the figures, identical reference numbers identify at least generally similar elements.

### DESCRIPTION

Embodiments disclosed herein may provide digital medicine support for predicting and mitigating side effects of bispecific antibody treatments (e.g., Pfizer's Elranatamab) for myeloma patients. The side effects may include cytokine release syndrome (CRS), infection (e.g., sepsis), neurotoxicity (e.g., peripheral neuropathy), cytopenia (e.g., neutropenia), etc. These side effects may be predicted based on passive collection of data from patient wearables and/or invisibles, patient entered data on healthcare application, and other data such as bloodwork data. Trained machine learning models may be used for predicting the side effects. As the side effects may be predicted before their onsets, a proactive intervention may be feasible to improve the healthcare outcomes for myeloma patients.

The predictions may be used for both in-hospital observation and at-home monitoring settings. For example, healthcare data collected leading up to and around the administration of bispecific antibody treatment may be used for predicting whether the patient may likely develop a CRS. The prediction may support a clinical decision as to whether the patient may be discharged for at-home monitoring (e.g., because of a low likelihood of the CRS) or whether the patient should be kept at the hospital for observation (e.g., because of the high likelihood of the CRS). During at-home monitoring, a prediction of one or more disease conditions may be made based on the passively collected data, patient entered data, and/or other data such as bloodwork data. If an adverse condition is predicted, alert notifications may be triggered to the patient and/or the clinician for a timely intervention to mitigate the adverse condition.

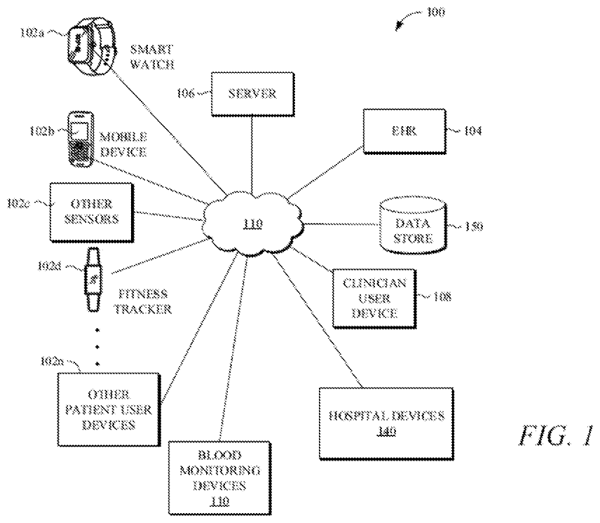

FIG. 1 shows a block diagram of an illustrative operating environment 100 for employing one or more embodiments of this disclosure. It should be understood that this and other arrangements described herein are set forth only as examples. Other arrangements and elements (e.g., machines, interfaces, functions, orders, and groupings of functions) can be used in addition to, or instead of, those shown in FIG. 1 as well as other figures, and some elements may be omitted altogether for the sake of clarity. Further, many of the elements described herein are functional entities that may be implemented as discrete or distributed components or in conjunction with other components, and in any suitable combination and location. Various functions or operations described herein are being performed by one or more entities including a hardware, firmware, software, and a combination thereof. For instance, some functions may be carried out by a processor executing instructions stored in memory.

As shown, the operating environment 100 may include patient facing user devices 102a-102n (collectively referred to as devices 102 or commonly referred to as device 102), blood monitoring devices 110, a server 106, an electronic health record (EHR) system 104, network 110, hospital devices 140, a data store 150, and a clinician user device 108. It should also be understood that the singular or plural description of the devices are just for the sake of clarity in explanation and should not be considered limiting. For instance, the server 106 may include multiple servers and the clinician user device 108 may include multiple user devices. The different devices in the operating environment 100 may be interconnected through the network 110.

The patient facing user devices 102 may include any type of computing and/or sensing device that the patients may interact with. The non-limiting examples shown in the operating environment 100 may include a smartwatch 102a, a mobile device 102b (e.g., a smartphone), other smart sensors 102c (e.g., a smart ring, invisible sensors such as motion sensors, etc.), a fitness tracker 102d, and other patient facing user devices 102n (e.g., tablets, laptop computers, desktop computers, smart speakers, smart home systems, etc.). The patient facing user devices 102 may either passively collect or actively prompt a patient to enter data associated with management of side effects associated with bispecific antibody treatments.

For example, the smartwatch 102a may have multiple sensors to passively collect data from the patient. The multiple sensors may include accelerometers, gyroscopes, and/or other types of motion sensors that may track the physical activity of the patient. The smartwatch 102a may further include sensors to detect biological parameters such as body temperature, heart rate, blood glucose level, blood oxygen saturation level, and/or any other type of biological parameters. The biological parameters may be continuously and/or periodically collected by the smartwatch 102a—e.g., without the patient explicitly involved in the collection—and provided to other components (e.g., server 106) in the operating environment.

The mobile device 102b may be used by the patient for actively entering health related data. For example, the mobile device 102d may be a smartphone that may have a healthcare application, e.g., an electronic patient reported outcome (ePRO) application, installed therein. The healthcare application may prompt the patient to enter health related data. For instance, the healthcare application may generate a push notification for the patient to enter data as to how the patient is feeling at the given point in time. The prompt may be, “How are you feeling this morning?” and the patient may enter, “I am feeling great.” The healthcare application may also display alert notification, which may be generated when the operating environment 100 determines that there is a likely risk of CRS, cytopenia, neutropenia, etc. Another type of alert notification displayed by the healthcare application may be when the current disease behavior of the patient deviates significantly from the established baseline behavior. In response, the healthcare application may further allow the patient to communicate, synchronously or asynchronously, with the clinician.

The other sensors 102c may include devices such as smart rings, skin patches, ingestible sensors, and/or any other type of body attached or non-body attached sensors (generally referred to as invisibles or invisible devices/sensors). The other sensors 102c may detect biological or non-biological data. For instance, other sensors 102c may include a smart fabric measuring a body temperature of the patient. As another example, the other sensors 102c may include a smart home sensor measuring home temperature and/or humidity. As yet another example, the other sensors 102c may include a motion sensor that may detect/measure movement within a room. The other patient devices 102n may include any other type of device associated with the patients. For instance, the other patient devices 102n may include tablet computers, laptop computers, desktop computers, and/or any other computing devices associated with the patients and connected to the network 110.

The blood monitoring devices 110 may include any kind of blood monitoring device and/or service that may collect and analyze blood samples of the patient. For example, the blood monitoring devices 110 may include a home mailed blood collection kit that may allow the patient to draw blood and send the kit back to lab for blood analysis. In other instances, the blood monitoring device 110 may comprise home mailed blood collection kit that may perform at least a portion of the blood analysis itself and provides the result of the analysis at one or more of the patient facing devices 102 and/or the server 106. The blood monitoring devices 110 should further be understood to include monitoring devices used by clinicians in a lab setting or in a home visit setting. Therefore, any type of technology and/or service used for collecting patient blood samples and analyzing the samples of biological parameters should be considered within the scope of this disclosure. An example of the biological parameter measured by the blood monitoring devices 110 may include white blood cell count, wherein a lower white blood cell count may indicate an onset of cytopenia.

The network 110 may include any kind of communication network. For instance, the network 110 may include packet switching network supporting protocols such as TCP/IP. The network 110 may also include circuit switching networks supporting both wired and wireless telephony. The network 110 therefore may include components such as wires, wireless transmitters, wireless receivers, signal repeaters, signal amplifiers, switches, routers, communication satellites, and/or any other type of network and communication devices. Some non-limiting examples of the network 110 may include local area network (LAN), metropolitan area network (MAN), wide area network (WAN) such as the Internet, etc. These are just but a few examples of the network 110, and any kind of communication linkage between the different components of the operating environment 100 are to be considered within the scope of this disclosure.

The server 106 may include any type of computing devices that may provide the analytical functionality of training and deploying one or more machine learning models and/or establishing and deploying statistical analytic models. For instance, the server 106 may train a prediction model for predicting CRS progression, e.g., whether a patient receiving a bi-specific antibody treatment will develop a CRS, and/or how likely is it that a patient receiving a bi-specific antibody treatment will develop a CRS. The prediction model may be trained using a supervised training approach, with labeled ground truth data. The prediction model may include, for example, regression model, a gradient boosted regression model, a logistic regression model, a random forest regression model, an ensemble model, a classification model, a deep learning neural network, a recurrent neural network for deep learning, or a convolutional neural network for deep learning. The same prediction model or another prediction model may be trained to determine whether the patient may develop other adverse health conditions.

The machine learning based prediction models described above are just examples and other statistical models are to be considered within the scope of this disclosure. For instance, the server 106 may also establish an analytical model based on the continuously collected longitudinal healthcare data, wherein the analytical model may indicate a baseline healthcare behavior. When new healthcare data is received, the server 106 may compare the received data with the analytical model (e.g., against the baseline healthcare behavior) to determine whether the new healthcare data shows a significant deviation from the baseline health behavior. The server 106 may also generate one or more alert notifications, e.g., to patients and/or clinicians, indicating that the patient is likely to develop one or more adverse conditions (e.g., CRS, cytopenia, neutropenia, etc.) and/or that the patient's health behavior has significantly deviated from the baseline behavior.

The electronic health record (EHR) 104 may store the health records of the patients. The health records may include, for example, the patients' ongoing condition (e.g., myeloma), prescribed medications, summaries of clinical encounters, and/or any other healthcare related data associated with the patients. In some embodiments, the EHR 104 may be maintained by healthcare providing entity (e.g., a hospital system).

The data store 150 may include any kind of database storing data collected from various sources within the operating environment 100. For instance, the data store 150 may store data collected, both passively and actively, from the patient facing devices 102. The data store 150 may also store data collected from blood monitoring devices 110. Additionally, the data store 150 may store data sourced from EHR 104. Therefore the data source 150 should be understood to store any kind of data in the operating environment 100.

The clinician user device 108 may be any kind of computing device showing a clinician dashboard. Non-limiting examples of the clinician user device 108 may include a mobile phone (e.g., a smartphone), a tablet computer, a laptop computer, a desktop computer, and/or any other type of computing device. The clinician dashboard may show information (e.g., demographic information, location information) and/or one or more alerts associated with the various patients.

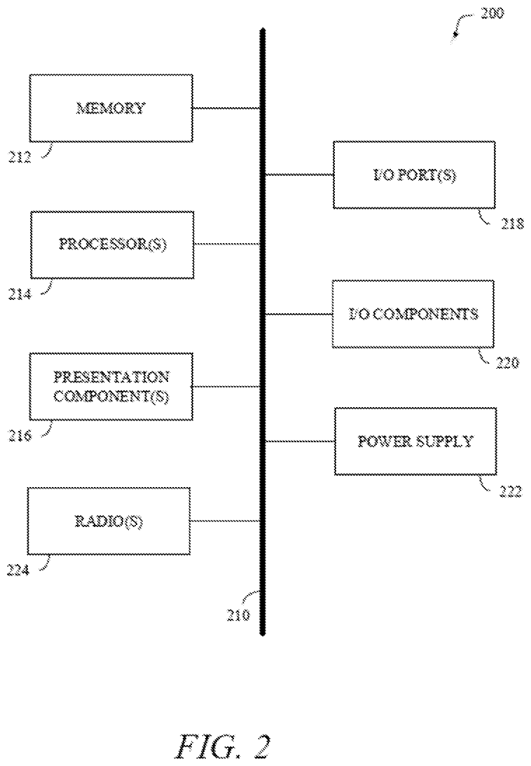

FIG. 2 shows a block diagram of an illustrative computing device 200 that may perform one or more functions described herein, according to the several embodiments of this disclosure. The computing device 200 is but one example of a suitable computing environment and is not intended to suggest any limitation as to the scope of use or functionality of embodiments of the disclosure. Neither should the computing device 200 be interpreted as having any dependency or requirement relating to any one or combination of components illustrated.

Embodiments of the disclosure may be described in the general context of computer code or machine-useable instructions, including computer-useable or computer-executable instructions, such as program modules, being executed by a computer or other machine, such as a personal data assistant, a smartphone, a tablet PC, or other handheld or wearable device, such as a smartwatch. Generally, program modules, including routines, programs, objects, components, data structures, and the like, refer to code that may perform particular tasks or implement particular data types. Embodiments of the disclosure may be practiced in a variety of system configurations, including handheld devices, consumer electronics, general-purpose computers, or more specialty computing devices. Embodiments of the disclosure may also be practiced in distributed computing environments where tasks are performed by remote-processing devices that are linked through a communications network. In a distributed computing environment, program modules may be located in both local and remote computer storage media including memory storage devices.

The computing device 200 may include a bus 210 that may directly or indirectly couple the following example devices: memory 212, one or more processors 214, one or more presentation components 216, one or more input/output (I/O) ports 218, one or more I/O components 220, and a power supply 222. Some embodiments of computing device 200 may further include one or more radios 224. Bus 210 represents what may be one or more busses (such as an address bus, data bus, or combination thereof). Although the various blocks of FIG. 2 are shown with lines for the sake of clarity, these blocks may represent logical, and not necessarily actual, components. For example, one may consider a presentation component 216 such as a display device to be an I/O component. Also, processors 214 may have their memories. Furthermore, distinction is not made between such categories as “workstation,” “server,” “laptop,” or “handheld device,” as all are contemplated within the scope of FIG. 2 and with reference to “computing device.”

The computing device 200 may include a variety of computer-readable media. Computer-readable media can be any available media that can be accessed by computing device 200 and may include both volatile and nonvolatile, removable and non-removable media. By way of example, and not limitation, computer-readable media may comprise computer storage media and communication media. Computer storage media may include both volatile and nonvolatile, removable and non-removable media implemented in any method or technology for storage of information such as computer-readable instructions, data structures, program modules, or other data. Some non-limiting examples of computer readable media may include RAM, ROM, EEPROM, flash memory or other memory technology, CD-ROM, digital versatile disks (DVDs) or other optical disk storage, magnetic cassettes, magnetic tape, magnetic disk storage or other magnetic storage devices, or any other medium which can be used to store the desired information and which can be accessed by computing device 200. Communication media may embody computer-readable instructions, data structures, program modules, or other data in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, communication media may include wired media, such as a wired network or direct-wired connection, and wireless media, such as acoustic, RF, infrared, and other wireless media. Combinations of any of the above should also be included within the scope of computer-readable media.

The memory 212 may include computer storage media in the form of volatile and/or nonvolatile memory. The memory 212 may be removable, non-removable, or a combination thereof. Some non-limiting examples of hardware devices for the memory 212 include solid-state memory, hard drives, optical-disc drives, etc.

The computing device 200 may include one or more processors 214 that read data from various entities such as memory 212 or the I/O components 220. The presentation component(s) 216 may present data indications to a user or other device. Exemplary presentation components may include a display device, speaker, printing component, and the like.

The I/O ports 218 may allow computing device 200 to be logically coupled to other devices, including I/O components 220, some of which may be built in. Non-limiting examples of the I/O components 220 may include a microphone, joystick, game pad, satellite dish, scanner, printer, or a wireless device. The I/O components 220 may provide a natural user interface (NUI) that processes air gestures, voice, or other physiological inputs generated by a user. In some instances, inputs may be transmitted to an appropriate network element for further processing. A NUI may implement any combination of speech recognition, touch and stylus recognition, facial recognition, biometric recognition, gesture recognition both on screen and adjacent to the screen, air gestures, head and eye tracking, and touch recognition associated with displays on the computing device 200. The computing device 200 may be equipped with cameras, such as stereoscopic camera systems, infrared camera systems, RGB camera systems, and combinations of these, for gesture detection and recognition. Additionally, the computing device 200 may be equipped with accelerometers or gyroscopes that enable detection of motion.

Some embodiments of computing device 200 may include one or more radio(s) 224 (or similar wireless communication components). The radio may transmit and receive radio or wireless communications. The computing device 200 may be a wireless terminal adapted to receive communications and media over various wireless networks. Computing device 200 may communicate via wireless protocols, such as code division multiple access (“CDMA”), global system for mobiles (“GSM”), or time division multiple access (“TDMA”), as well as others, to communicate with other devices. The radio communications may be a short-range connection, a long-range connection, or a combination of both a short-range and a long-range wireless telecommunications connection.

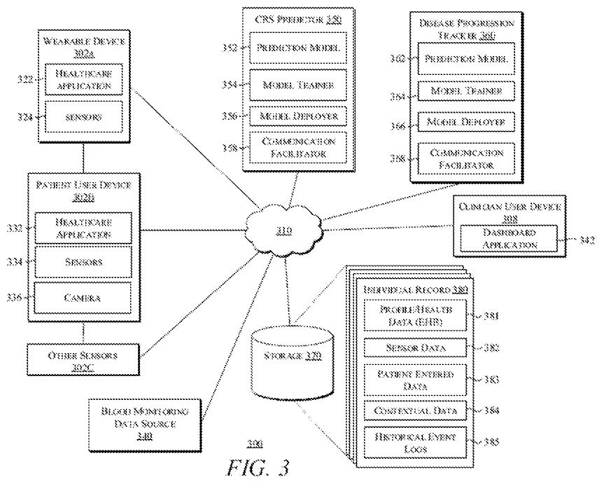

FIG. 3 shows a block diagram of an illustrative architecture of operating environment 300, wherein one or more embodiments disclosed herein may be employed. The shown architecture may be implemented by one of more components/devices (e.g. computing device 200 shown in FIG. 2) of the operating environment 100 shown in FIG. 1. It should however be understood that the shown architecture is just but an example and architectures with additional, alternative, or fewer number of components should also be considered within the scope of this disclosure. It should further be understood that components shown as a single components or plural components are also just examples: a single component may include multiple iterations of the same component or multiple constituent sub-components and the functionality of plural components may be achieved by a single component.

The operating environment 300 may be used for a continuous or near-continuous digital monitoring of patients undergoing bispecific antibody treatments (e.g., Pfizer's Elranatamab) and triggering alert notifications to the patients and/or clinicians as needed. Furthermore, the operating environment 300 may provide a seamless and continuous connectivity between the patients and clinicians. This continuous or near-continuous monitoring may allow for a proactive management of the side effects associated with the bispecific antibody treatments-a machine learning model may predict onset of one or more conditions and therefore may facilitate early interventions against those conditions by the clinicians. The operating environment 300 may further be used for clinical decision support, e.g., whether to discharge patient for at-home monitoring or whether to keep the patient at a hospital setting for observation after a bispecific antibody treatment session. To implement these and other functions, the operating environment 300 may include patient facing devices (e.g., wearable device 302a, patient user device 302b, other sensors 302c, etc.) to gather healthcare data and other data from the patients, to provide the alert notification to the patients and/or clinicians, and to facilitate communication between clinicians and patients. The data gathered from the patient facing devices and other sources (e.g., blood monitoring data source 340) may be stored in the storage 370 (e.g., as individual records 380). The analysis components (e.g., CRS predictor 350, disease progression tracker 360) may use the stored data and other data to predict CRS and/or track the progression of side effects (e.g., infections, neurotoxicity, cytopenia, etc.) associated with bispecific antibody treatment of myeloma. Based on the analysis, alert notifications may be sent to clinicians (e.g., through a clinician user device 308). The components of the operating environment 300 may be interconnected through a network 310.

With regard to the patient facing devices 302, these devices may include, for example, the wearable device 302a, the patient user device 302b, and other sensors 302c. The wearable device 302a may include any kind of wearable device: non-limiting examples include a smartwatch, a fitness tracker, a smart ring, etc. In some embodiments, the wearable device 302a may include a healthcare application 322. The wearable device 302a or any application installed thereon (e.g., healthcare application 322) may be a medically prescribed component within the operating environment 300. The healthcare application 322 may a computer program installed on the wearable device 302a to collect healthcare data, perform pre-processing of the collected data in some instances, and transmit the data to the patient user device 302b or to a remote server (e.g., a server implementing one or more of the CRS predictor 350, disease progression tracker 360, and/or storage 370). Particularly, the healthcare application 322 may interface with the operating system of the wearable device (e.g., through API calls) to gather the data from the sensors 324.

The sensors 324 may include any type of sensors that may continuously or periodically gather data from the patient wearing the wearable device 302a. For example, the sensors 324 may include biological sensors, such as a temperature sensor to measure the body temperature (it should be understood that the temperature sensor may be non-biological and may measure the ambient temperature), heart rate monitor, an electrocardiogram sensor to collect electrocardiogram data when prompted by the patient, a glucose monitor, a sweat monitor, blood oxygen saturation level monitor, blood pressure monitor, and/or any other type biological sensors. The sensors 324 may also include accelerometers to determine directional movement, gyroscopes to detect the orientation, and/or any other type of sensors gather positional or movement data. These sensors 324 may be triggered by the healthcare application 322 (e.g., through API calls to the operating system of the wearable device 302a) to collect the corresponding data. Alternatively, the wearable device 302a may not have the healthcare application 322 and the triggering may be received from the patient user device 302b (e.g., from its healthcare application 332) or remotely through the network 310. In other embodiments, the wearable device 302a may itself be continuously or periodically activating the sensors 324 and may pass the collected sensor data along to the healthcare application 324 (and/or healthcare application 332 or a remote device connected via the network 310). The biological data and the movement data collected by the sensors 324 may be collectively or commonly referred to as healthcare data (or health data).

In other words, the sensors 324 may be collecting healthcare data passively, i.e., without an active involvement of the patient. For instance, the sensors 324 may be monitoring the patient's biological data and/or physical movements because the sensors 324 are within the wearable device 302a and therefore are continuously attached to the patient. This passive collection of the healthcare data does not require the patient's continuous attention of the patient and is therefore less burdensome.

The patient user device 302b may include any kind of computing device used by the patient. For example, the patient user device 302b may include a mobile phone such as a smartphone, a tablet device, a laptop computer, a desktop computer, and/or any other type of computing device. A healthcare application 332 (e.g., an ePRO) may be installed on the patient user device 302b. The healthcare application 332 should be understood to include a standalone application (e.g., a smartphone app) or a web-based application (e.g., accessed using a browser). It should further be understood that the healthcare application 332 may also be medically prescribed. The healthcare application 332 may provide an interface (e.g., a graphical user interface) for the patient to view alert notifications, communicate with a clinician, and/or actively enter healthcare data (e.g., how the patient is feeling at a particular point in time).

As an example, the healthcare application 332 may be used gather further information on a prediction based on the data collected by the sensors 324 of the wearable device. For instance, using the passively collected data, the disease progression tracker 360 may predict an onset of a side effect of bispecific antibody treatment. For example, the sensors 324 of the wearable device 302a may measure less body movement compared to an established baseline; and the disease progression tracker 360 may predict an onset of neurotoxicity. In response to this prediction, an alert notification (e.g., by communication facilitator 368) may be sent to the healthcare application 332. The alert notification may be, for example, “We have detected lower activity level? Are you feeling fatigued?” and prompt the patient to respond. For the response, the healthcare application 332 may provide choices such as “Extremely Fatigued,” “Moderately Fatigued,” or “Not Feeling Fatigued.” As another example, the sensors 324 of the wearable device 302a may measure a lower than usual heart rate. In response to this determination, the server may transmit another alert notification to the healthcare application 332. The alert notification may include, for example, “We have detected a lower heart rate. Do you feel lightheaded?” and prompt the patient to respond. For the response, the healthcare application 332 may provide choices such as “Extremely Lightheaded,” “Moderately Lightheaded,” or “No Lightheadedness.” This are just but a few examples of the alert notifications to the patient and other types of alert notification should also be considered within the scope of this disclosure.

In addition to the prompts corresponding to alert notifications, the healthcare application 332 may request the user to periodically (e.g., without a trigger for data entry) enter healthcare data. For instance, the healthcare application 332 may prompt the patient to enter how they feel every morning, daytime, and evening. Other non-limiting examples of the actively entered data include body temperature (if not collected by the sensors 324 of the wearable device 302a), bowel movement, level of pain experienced, stress level, anxiety level, the time of intake of a prescription medication, exercise activity (if not captured by the wearable device 302a), observed side effects of the bispecific antibody treatment, and/or any other type of healthcare data.

In addition to the alert notifications, the healthcare application 332 may also provide two-way connectivity between the patient and the clinician. The two-way connectivity between the patient and the clinician may be facilitated by one or more of the communication facilitator 358 of the CRS predictor or the communication facilitator 368 of the disease progression tracker 360. The two-way connectivity may allow the patient to establish any time of synchronous (e.g., voice or a video call) or asynchronous (e.g., message exchanges) with the clinician. The two-way connectivity may be initiated in response to an alert notification. For instance, the disease progression tracker 360 may predict an onset of cytopenia and the corresponding communication facilitator 368 may have generated an alert provided on the healthcare application 332. When the patient selects the alert notification (e.g., a notification badge), an interface may be provided by the healthcare application for the patient to initiate a communication session with the clinician (e.g., messaging or calling the clinician). For instance, the patient may pose questions through the healthcare application 332 itself and the clinician's response may be displayed within the healthcare application 332. The two-way connectivity may also be leveraged to provide educational materials to the patient. The educational material in some embodiments may include cognitive behavior therapy (CBT) based behavior modification encouragement materials. These materials may be provided to the patient based on the healthcare data passively collected by the wearable device 302a, actively collected by the patient user device 302b, and analyzed by the disease progression tracker 360. The CBT-based materials may be in the form of audio, video, and/or text and encourage the patient to make healthier choices on food, rest and exercise, stress management, and/or any other metric associated with maintaining a good quality of life while undergoing bispecific antibody treatment.

The sensors 334 in the patient user device 302b may include any type of sensors such as heart rate sensors (e.g., when the patient brings the patient user device 302b closer to the body), glucose monitors (e.g., using an infrared camera), accelerometers, gyroscopes, etc. Generally, any type of biological and/or movement sensor should be considered within the scope of this disclosure. For example, in case of the patient user device 302b being a mobile device (e.g., a smartphone), the patient user device 302b too may monitor the user's movement using the sensors 334. The sensors 334 may detect the number of steps taken by the patient throughout the day and/or other activities (e.g., exercise) performed by the patient throughout the day. In other words, the sensors 334 too may be used to passively collect movement data of the patient. In some embodiments, the sensors 334 may enable an active data collection. For instance, the sensors 334 may include an infrared camera and the patient user device 302b may prompt the patient to hold their finger against the camera to detect biological attributes such as blood glucose level, blood oxygen saturation level, etc. In another example, the sensors 334 may include a heart rate sensor, which when brought close to the patient's body, may measure the patient's heart rate.

The camera 336 may include an optical camera that the patient may use to take healthcare related pictures. The picture may be of a relevant body part, e.g., picture of the patient's hand showing the state of the skin. The pictures may be sent to the storage 370 and/or provided to the clinician. The clinician may use the pictures for diagnostic purposes (e.g., to determine whether a particular side effect is improving or worsening) and/or for therapeutic purposes (e.g., to determine whether to adjust the dosage the medication the patient is taking.)

The other sensors 302c may be any kind of sensors measuring one or more biological or physical attributes of the patient. An example of the other sensors 302c may be an ingestible sensor that may be measure the effect on gut activity of the bispecific antibody treatment and/or other associated prescription medications. Another example may be a patch sensor that may be attached to the skin to measure attributes such skin temperature and/or the movement of the body part that the patch sensor is attached to. The sensors 302c may further include a blood pressure monitor that may communicate measurements to the patient user device 302b or any other device within the operating environment 300. Other examples of the sensors 302c may include smart fabrics, smart belts, sub-cutaneous sensors, etc. These are just but a few examples of the sensors and any type of body-worn or non-body-worn sensor should be considered within the scope of this disclosure. The non-body worn sensors may be referred to as invisibles or invisible sensors/devices.

The blood monitoring data source 340 may be any kind of blood analysis device or system. For example, the blood monitoring data source 340 may include at home blood collection kit. The at home blood collection kit may be mailed to the patient's home and used by the patient to collect blood specimen to be sent to a lab. The lab may in turn perform the blood analysis (e.g., determine white blood cell count) and provide the analysis data through the network 310 to other components (e.g., storage 370) of the operating environment 300. In other embodiments, the home collection kit may have some blood analysis capacity in combination of the patient user device 302b—e.g., an analysis component in the kit may connect with the patient user device 302b to provide the measured data.

The blood monitoring data source 340 may further include other types of sources such as labs, doctor's offices, and/or any type of equipment and/or establishment for collecting and analyzing blood samples. Regardless of its type, the blood monitoring data source 340 may provide the measured data to other components within the operating environment 300 (e.g., through the network 310).

As described above, the data collected—either passively or actively—by one or more of the wearable device 302a, patient user device 302b, other sensors 302c, and the blood monitoring data source 340 may be received by other components in the operating environment through the network 310. The network 310 may include any kind of communication network. For instance, the network 310 may include packet switching network supporting protocols such as TCP/IP. The network 310 may also include circuit switching networks supporting both wired and wireless telephony. The network 310 therefore may include components such as wires, wireless transmitters, wireless receivers, signal repeaters, signal amplifiers, switches, routers, communication satellites, and/or any other type of network and communication devices. Some non-limiting examples of the network 310 may include local area network (LAN), metropolitan area network (MAN), wide area network (WAN) such as the Internet, etc. These are just but a few examples, and any kind of communication linkage between the different components of the operating environment are to be considered within the scope of this disclosure.

The data received from the patient facing devices (e.g., wearable device 302a, patient user device 302b, other sensors 302c, etc.) and other sources (e.g., blood monitoring data source 340) may be stored in the storage 370. The storage 370 may include any kind of storage technology such as hard drive storage, solid state storage, data server storage, etc. Although a single storage 370 is shown for the clarity of explanation, the storage 370 should be understood to include multiple, geographically distributed components. For example, the storage 370 may be distributed among multiple data centers and incorporate multiple levels of redundancies.

In some embodiments, the storage 370 may store individual records 380 containing the data for the corresponding patients. In other words, an individual record 380 may be associated with the patient. It should however be understood that this individual record 380-based organization of data is just an example and should not be considered limiting. Any kind of data organization (e.g., relational, object oriented) should be considered within the scope of this disclosure.

As shown, an individual record 380 of a patient may include a profile/health data (e.g., electronic health record (EHR) data) 381, sensor data 382, patient entered data 383, contextual data 384, and historical event logs 385. However, these are just some examples of the pieces of data types within the individual record 380; and additional, alternative, or fewer pieces of data types should also be considered within the scope of this disclosure. Furthermore, the discrete data types shown herein are just examples as well, and a data type may include aspects of other data types. For example, the profile/health data may incorporate historical event logs 385.

The profile/health data 381 may include the electronic health record of the corresponding patient. The profile/health data 381 may therefore include a demographic information, comprehensive medical history, family medical history, allergies, ongoing conditions, records of clinical encounters, other notes from clinicians, prescription medications, laboratory results, and/or any other type of healthcare data for the patient. For example, the profile/healthcare data 381 may include information about the treatment regime using a bispecific antibody and any observed side effects of administrating the bispecific antibody. In some embodiments, the profile/health data 381 may be sourced to the storage 370 from other entities. For instance, the profile/health data 381 may be managed by a healthcare providing entity (e.g., a hospital), and the operating environment 300 may retrieve the data from the healthcare providing entity.

The sensor data 382 may be the data from the patient facing sensors such as the sensors 324 of the wearable device 302a, sensors 334 of the patient user device 302b, and/or other sensors 302c. The sensor data 382 may therefore include data from biological sensors (e.g., heart rate monitors, blood pulse oximeters), the movement sensors (e.g., accelerometers and/or gyroscopes), and/or any other type of sensors. The sensor data 382 may be stored in association with the timestamps of when the data was collected. The timestamps may allow the operating environment 300 to detect the patient's activity and condition throughout the day. As used herein, the sensor data 382 should be generally understood to include any kind of passively collected data (e.g., movement passively detected by a wearable), or data captured by the patient actively engaging with the sensor (e.g., the patient putting their finger on an infrared camera to measure various biological attributes).

The patient entered data 383 may include any kind of data actively entered by the patient (e.g., through the healthcare application 332). The patient entered data 383 may therefore include, the patient's entry as to how they have been feeling (e.g., “Fatigued,” “Depressed,” “Fine,” etc.) at a particular point in time. As another example, the patient entered data 383 may include the patient's response to various alert notifications provided by the healthcare application 332. The patient entered data 383 may further include other biological data not captured by the sensors (e.g., sensors 324, 334, and/or 302c). For instance, such biological data may include blood glucose level, blood oxygen saturation level, blood pressure, etc. captured by devices (e.g., an external blood pressure monitor) operating with active user engagement within the operating environment 300. As with the sensor data 382, the patient entered data 383 may also be organized using the timestamps. In other words, the timestamps may be used to correlate the sensor data 382 and the patient entered data 383.

The contextual data 384 may include any kind of information that may provide more context to the sensor data 382 and/or the patient entered data 383. For example, the contextual data may include data from the blood monitoring data source 340. As another example, contextual data may include geographical information about the patient (e.g., which region of the country the patient resides), information about the disease condition of a cohort similar to the patient. Generally, any type of information that generates additional data points within the operating environment 300 should be considered as contextual data 384. As with the sensor data 382 and the patient entered data 383, the contextual data 384 may also be timestamped, such that these three types of data may be temporally correlated during further analysis (e.g., by CRS predictor 350 and/or the disease progression tracker 360).

The historical event logs 385 may include a record of events associated with the patient. For instance, the historical event logs 385 may include other information on clinical encounters, prescription filling and refilling, and/or any other types of events associated with managing side effects of bispecific antibody treatments. The historic event logs 385 may also be timestamped such that these logs can be temporally correlated with one or more of the sensor data 382, patient entered data 383, or contextual data 384.

The analytic components (e.g., CRS predictor 350 and disease progression tracker 360) may use the individual records 380 in the storage 370 (and/or other types of data) to generate/train one or more machine learning models (and/or any other type of analytic models), and then deploy the trained models for one or more of predicting an onset of CRS and/or onset of other side effect associated with bispecific antibody treatments.

The CRS predictor 350 may predict a likelihood of a CRS in a patient undergoing bispecific antibody treatment. Such prediction may be based on using a prediction model 352, which may be trained by a model trainer 354 and deployed by a model deployer 356. The prediction model may include, for example, regression model, a gradient boosted regression model, a logistic regression model, a random forest regression model, an ensemble model, a classification model, a deep learning neural network, a recurrent neural network for deep learning, or a convolutional neural network for deep learning.

The model trainer 354 may train the prediction model using a training dataset. To that end, the model trainer 354 may include computer program instructions that may retrieve the training data set, pre-process the training data set, and use the training data to train the prediction model 352, e.g., using one or more of supervised or unsupervised training approaches. In an example training using a supervised training approach, the model trainer 354 may retrieve a labeled training dataset. The labeling may have been done by humans based on past observations. For instance, the labeled dataset may have a set of healthcare data inputs, such as data collected passively by wearable sensors, invisible sensors, etc., collected with active patient engagement on the applications running the patient user devices, and/or data of laboratory results (e.g., bloodwork). These inputs may be associated with observed outputs—for example, if a patient associated with a first set of inputs developed CRS, that data may be human labeled as being associated with CRS. Similarly, if a patient associated with a second set of inputs did not develop CRS, the corresponding data may be human labeled as not being associated with CRS.

Therefore, using the labeled dataset (e.g., where the ground truth of the output is provided with the inputs), the model trainer 354 may train the prediction model 352. However, it should be understood that the supervised training approach is just but one approach and should not be considered limiting. The model trainer 354 may use other approaches, e.g., unsupervised training approach to train the model.

Although the prediction model 352 is described herein as a machine learning model, it should be understood that the prediction model 352 may include a statistical model. For the statistical model, the model trainer 354 may function as a model generator to generate the statistical model. The statistical model may be used for predicting which combinations of input variables (e.g., healthcare data from various sources) may more likely result in a “CRS” output; and which combinations of the input variables may more likely result in a “no-CRS” output.

It should be understood that the model trainer 354 may continuously train the prediction model 352. For instance, if the ground truth is available for a prediction (e.g., the ground truth may indicate whether the prediction was correct or incorrect), the model trainer 354 may use such correct or incorrect prediction to continuously train and improve upon the prediction model 352.

The model deployer 356 may be software module using the trained prediction model 352 to predict the likelihood of a CRS from received input data. For example, a new healthcare data may be received for a patient undergoing bispecific antibody treatment. The new healthcare data may include, for example, passively collected data from wearable device 302a, data collected with patient engagement from the patient user device 302b, data collected from other sensors 302c, data collected from the blood monitoring data source 340, etc. The healthcare data may include, for example, heart rate, skin temperature, blood pressure, blood oxygen saturation, etc. When this healthcare data is fed into the trained prediction model 352, the prediction model may output a likelihood of CRS. The likelihood may be expressed as probabilistic output, e.g., 85% likely to develop CRS and 15% not likely to develop CRS. Alternatively or additionally, the likelihood may be expressed as a binary classification, e.g., “likely to develop CRS” and “not likely to develop CRS.” The binary classification may be driven by the underlying probabilistic output and may use a threshold. For instance, the binary classification may output “likely to develop CRS” when the probabilistic output crosses “55% likely to develop CRS” threshold. The threshold may be adjusted as the prediction model 352 is continuously trained and refined.

The communication facilitator 358 may generate one or more notifications based on the trained prediction model 352 indicating a higher likelihood of a CRS. A notification may be provided to the clinician dashboard 342. In some instances, the notification may provide the probabilistic likelihood of the patient developing CRS. In other instances, the notification may be binary output of whether or not the patient is likely to develop CRS. The dashboard application 342 may be showing a view of the patient's profile/health data 381 and the notification may be presented in the view. In addition to a notification to the clinician dashboard 342, the communication facilitator 358 may transmit another notification to the healthcare application 332 running on the patient user device 302b. The notification to the patient may not necessarily indicate a likelihood of CRS, but simply indicate that a clinician will be following up on healthcare matter. One or more of these notifications may also establish a two-way connectivity between the patient and the clinician.

Based on the output of the prediction model 352 and the subsequent notification to the clinician dashboard application 342, the clinician may determine whether the patient may be discharged for at-home monitoring or should be kept at the hospital for observation. For example, the clinician may determine that patients with a higher likelihood of developing a CRS should be kept at the hospital and those with a lower likelihood of developing CRS may be discharged for at-home monitoring.

The at-home monitoring may be driven by the disease progression tracker 360, which may be another type of analytics provided within the operating environment 360. Generally, the disease progression tracker may use the prediction model 362 (and/or a statistical model) to determine whether an at-home monitored patient may develop side effects such as CRS, infections, neurotoxicity, cytopenia, etc. To that end, the disease progression tracker 360 may use a model trainer 364 to train the prediction model 362, a model deployer 366 to deploy the prediction model 362, and a communication facilitator 368 to generate one or more notifications and/or establish two-way connectivity based on the output of the prediction model 362.

The model trainer 364 may include computer program instructions that may retrieve long term data from the storage (e.g., profile health data 381, historical event logs 385, etc.). This long-term data may be used to train the prediction model 362 (and/or generate an analytic model). The prediction model should be understood to include any type of machine learning model such as regression model, a gradient boosted regression model, a logistic regression model, a random forest regression model, an ensemble model, a classification model, a deep learning neural network, a recurrent neural network for deep learning, or a convolutional neural network for deep learning. The model trainer 364 may use a supervised training method, e.g., with labeled output data to train the prediction model 362. However, it should be understood that unsupervised approach may also be used to supplement the supervised training approach.

In some embodiments, the model generator 364 may generate models (e.g., train the prediction model or generate an analytical model) for individual patients. For instance, the model generator 364 may retrieve long-term data for individual patients and then establish a baseline as a corresponding trained prediction model 362. The baseline may include, for example, normal levels of physical activity, biological data (e.g., blood oxygen saturation level, etc.), the feelings reported by the patient (e.g., “feeling fatigued”). The combination of the normal levels of these attributes may therefore be established as a baseline in the trained prediction model 362 or the generated analytical model. In the deployment phase, the newly received healthcare data may be compared against the baseline to determine whether there is significant amount of deviation from the established baselines.

In some embodiments, the model generator 364 may generate population level baselines. For instance, the model generator 364 may retrieve long-term data for a population of patients with certain criteria, e.g., age, gender, geographical location, ethnicity, etc. Analyzing the collected data, the model generator 364 may establish a population level baseline in the trained prediction model 362 (and/or a statistical model). The model deployer 366 may later use the population level baseline to determine whether an individual patient's condition has deviated significantly from the normal level.

The communication facilitator 368 may facilitate a communication between the patient and the patient, e.g., by using the healthcare application 332 and the dashboard application 342. For example, of the analytics model 362 determines that the state of a condition has significantly deviated from normal and that the patient may likely develop a side effect (e.g., infection), the communication facilitator 368 may transmit a first alert notification to the patient (e.g., to be displayed on the healthcare application 332 of the patient user device 302b) and a second alert notification to the clinician (e.g., to be displayed on the dashboard application 342 of the clinician user device 308). One or more of these alerts may have a communication prompt. For instance, the first alert to the patient may have a prompt “Send A Message To My Doctor.” The second alert to the clinician may be “Reach Out to Patient A, He May Be Developing An Infection.” In response to these prompts, an asynchronous (e.g., through text message exchange) or synchronous (e.g., through audio/video chat) communication channel may be opened between the healthcare application 332 and the dashboard application 342. In addition to this two-way connectivity, the clinician may be able to perform other actions such as prescribe medications, provide educational materials, and/or any perform any other type of patient care actions.

The dashboard application 342 may be displayed on a clinician user device 308, which may be any kind of user device used by a clinician. Non-limiting examples of the clinician user device 308 may include mobile phone (e.g., smartphone), tablet computer, laptop computer, desktop computer, etc. The dashboard application 342 may be an installed stand-alone application or web-based application accessible through a browser. The dashboard application 342 may show the disease progress of an individual patient. For instance, the dashboard application 342 may show a chart showing how the biological data (e.g., blood oxygen saturation level) has changed over time. The dashboard application may also other aspects of the patient's health, e.g., levels of stress and anxiety, etc. The dashboard application 342 may further show the medications prescribed for the patient. Generally, the dashboard application 342 may show any type of clinical data and notifications for the patient being treated and monitored in in the operating environment 300.

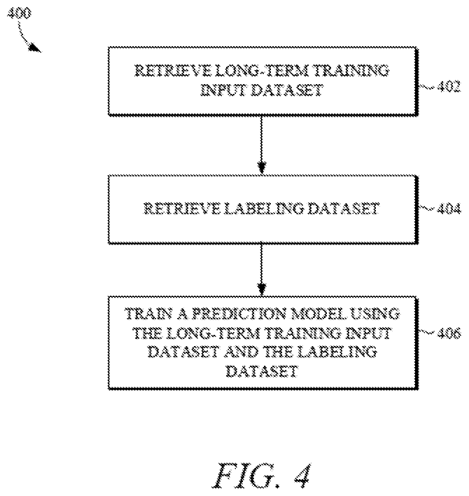

FIG. 4 shows a flow diagram of an illustrative method 400 of training a prediction model for CRS prediction, according to several embodiments of this disclosure. The illustrative method 400 may be implemented by one or more computing devices (e.g., computing device 200 as used in the operating environment 100). It should be understood that the steps of the method 400 shown in FIG. 4 and described herein are merely illustrative and methods with additional, alternative, or a fewer number of steps should be considered within the scope of this disclosure.

At step 802, long-term training input dataset may be retrieved. The long-term training dataset may include healthcare data for a population of patients that may have received bispecific antibody treatments. The healthcare data may include, for instance, passively collected biological data (e.g., temperature, blood oxygen saturation level, heartrate, blood pressure) from wearables, data collected through patient engagement in applications installed on patient devise, data from laboratory analysis (e.g., bloodwork) for the lead up to and around a bispecific antibody treatment session. In addition, the long-term training input dataset may include other information such as the patients' demographic information, medical history, family medical history, and/or any other healthcare attribute that may likely influence the bispecific antibody treatment. The dataset retrieved at this step may be used as inputs for training the prediction model.

At step 404, a labeling dataset may be retrieved. The labeling dataset may generally indicate whether patients associated with the long-term training input dataset actually developed a CRS. The labeling dataset may therefore come from various sources. For instance, the labeling dataset may be sourced from the patients' electronic health records (e.g., EHR 381 shown in FIG. 3). In some embodiments, the labeling dataset retrieved at step 404 may be used to manually label the long-term training input dataset retrieved at step 402.

Therefore, the combination of the long-term training input dataset and the labeling dataset may provide labeled training dataset for training a prediction model in step 406. Using the labeled training dataset, the prediction model may be trained with a supervised training approach. For instance, each training iteration may generate an output, which may be compared against the expected output (e.g., as shown by the labels), and backpropagation techniques may be used to refine the prediction model such that the prediction model generates an output closer to the expected output. Some non-limiting examples of the prediction model may include a regression model, a gradient boosted regression model, a logistic regression model, a random forest regression model, an ensemble model, a classification model, a deep learning neural network, a recurrent neural network for deep learning, or a convolutional neural network for deep learning.

This approach of training the prediction model is just an example and other approaches should also be considered within the scope of this disclosure. For example, the prediction model may be trained using an unsupervised training approach. In other examples, the prediction model may be a statistical model, and step 406 may establish the statistical model using the retrieved datasets. Therefore, any type of data analytics to generate a prediction model or to establish a statistical model should be considered within the scope of this disclosure.

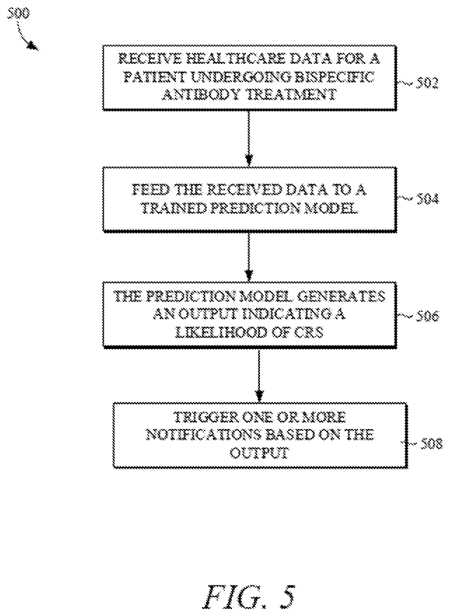

FIG. 5 shows a flow diagram of an illustrative method 500 of using a trained prediction model for CRS detection, according to some embodiments of this disclosure. The illustrative method 500 may be implemented by one or more computing devices (e.g., computing device 200 as used in the operating environment 100). It should be understood that the steps of the method 500 shown in FIG. 5 and described herein are merely illustrative and methods with additional, alternative, or a fewer number of steps should be considered within the scope of this disclosure.

At step 502, healthcare data for a patient undergoing bispecific antibody treatment may be received. Prior to receiving the treatment, the patient may be provided (e.g., based on a prescription) with a wearable device (e.g., a smartwatch) and/or a healthcare application installed on the patient's user device. The wearable device may passively capture data such as movement, blood oxygen saturation level, blood pressure, heart rate, body temperature, etc. The healthcare application may allow the patient to create a profile and enter other data such as medical history. Additionally, the healthcare data may include laboratory data (e.g., results of the bloodwork). The healthcare data collection may begin at a predetermined time before the administration of the bispecific antibody. For instance, the patient may be provided with the data collection devices (e.g., the wearable, the smartphone application) a few days before the administration of the bispecific antibody, and the data collection may continue during and after the administration.

At step 504, the received data may be fed to a trained prediction model. The predication model may have been trained using the steps of the method 400. The trained prediction model should be understood also to include any type of established statistical model. Some non-limiting examples of the prediction model may include a regression model, a gradient boosted regression model, a logistic regression model, a random forest regression model, an ensemble model, a classification model, a deep learning neural network, a recurrent neural network for deep learning, or a convolutional neural network for deep learning. In some cases, a statistical model may be used, where step 504 may include comparing a statistic (e.g., a z-statistic) to determine if the received data is significantly closer a likely outcome (e.g., an outcome indicating a CRS). It should however be understood that these are just examples and other prediction/statistical model should be considered within the scope of this disclosure.

At step 506, the prediction model (or a statistical model) generates an output indicating a likelihood of a CRS. The likelihood of CRS may indicate corresponding of probabilities of the fed inputs being associated with CRS and not being associated with CRS. For example, the output may be probability of a CRS 90% and probability of no-CRS 10%. In other embodiments, the outputs may be binary: for example, if the probabilistic likelihood crosses a certain threshold, the output may be likely CRS, and not likely CRS otherwise.

At step 508, one or more notifications may be triggered based on the output of the prediction model. For example, if the prediction model generates a higher likelihood of CRS, a notification may be triggered to the clinician's dashboard. The notification may indicate that a certain patient may likely develop a CRS. This notification may provide a clinical decision support to the clinician whether to discharge the patient for at-home monitoring or keep the patient at the hospital for observation. For instance, the clinician may recommend at-home monitoring for patients with lower likelihood of CRS and recommend in-hospital observation for patients with a higher likelihood of CRS. In addition to the notification to the clinician, notification to the patients may also be generated. A notification to the patient may include that the clinician will reach out the patient with important clinical information.

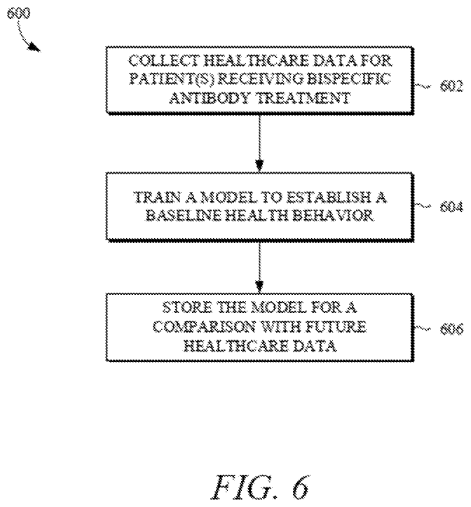

FIG. 6 shows a flow diagram of an illustrative method 600 of training a model to establish a baseline health behavior, according to the several embodiments of this disclosure. The illustrative method 600 may be implemented by one or more computing devices (e.g., computing device 200 as used in the operating environment 100). It should be understood that the steps of the method 600 shown in FIG. 6 and described herein are merely illustrative and methods with additional, alternative, or a fewer number of steps should be considered within the scope of this disclosure.

At step 602 healthcare data from one or more patients receiving bispecific antibody treatment may be received. The healthcare data may include data passively collected data from a wearable device (e.g., a smartwatch) and/or invisibles, and/or actively collected through a healthcare application (e.g., installed on a smartphone). The passively collected data may include, for example, biological data (e.g., heart rate) and movement data. The actively collected data may include, for example, the state of the patient's feeling, the prescription medication being taken, and/or other biological data entered by the patient. Another type of healthcare data may include bloodwork data.

The collected data may therefore be a longitudinal dataset indicating the progress of different conditions (e.g., infections) associated with the bispecific antibody treatment. Accordingly, this collection may be used at step 604 to train a model to establish a baseline health behavior. A baseline health behavior may indicate, for example, a normal distribution of a combination of variables such as biological parameters, the patient's state of feelings (e.g., stress and anxiety levels), patient's activity level, bloodwork analysis, and/or any other attribute associated with the ongoing conditions. The baseline may be established on an individual patient level and/or may be established at a cohort population level. Non-limiting examples of the trained model may include, regression model, a gradient boosted regression model, a logistic regression model, a random forest regression model, an ensemble model, a classification model, a deep learning neural network, a recurrent neural network for deep learning, or a convolutional neural network for deep learning.

At step 606, the trained model may be stored for comparison with future healthcare data. As the healthcare data is collected on an ongoing basis, such comparison may allow a determination whether the future collected healthcare data is within an expected distribution range or deviates significantly from the expected distribution range. A significant deviation from the expected distribution range may indicate that a clinical intervention is likely needed.

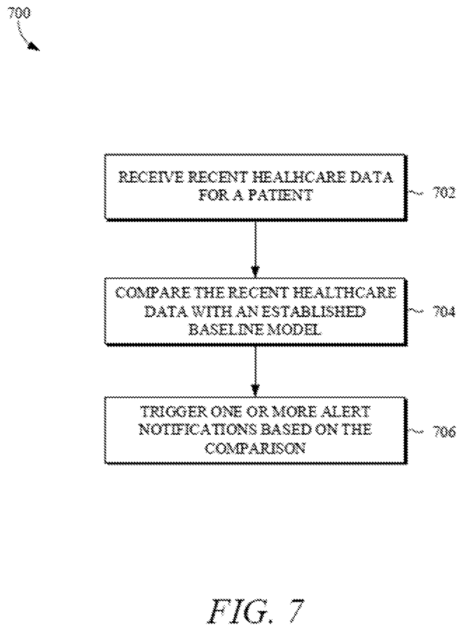

FIG. 7 shows a flow diagram of an illustrative method 700 of deploying a baseline model to determine whether the current observed behavior deviates significantly from the baseline disease behavior, according to several embodiments of this disclosure. The illustrative method 700 may be implemented by one or more computing devices (e.g., computing device 200 as used in the operating environment 100). It should be understood that the steps of the method 700 shown in FIG. 7 and described herein are merely illustrative and methods with additional, alternative, or a fewer number of steps should be considered within the scope of this disclosure.

At step 702, recent healthcare data for a patient (e.g., a patient undergoing bispecific antibody treatment) may be received. The recent healthcare data may be collected passively, e.g., through wearables or invisibles; actively, e.g., prompting the patient to enter data in a healthcare application; and/or through other systems such as blood collection and analysis systems. Such recent healthcare data may be gathered continuously, as the patient may be monitored in an ongoing basis (e.g., at-home monitoring).

At step 704, the recent healthcare data may be compared with an established baseline model (e.g., baseline model generated by method 600). The comparison may include determining whether the recent healthcare data shows a statistically significant deviation from an established baseline health behavior (as indicated by the baseline model).

At step 706, one or more notifications may be triggered based on the comparison. For instance, if it is determined that the patient healthcare behavior has significantly deviated from the established healthcare behavior, an alert notification may be sent to the patient to initiate a synchronous or asynchronous communication with a clinician. Additionally or alternatively, another alert notification may be sent to a clinician to initiate a synchronous or asynchronous communication with the patient.

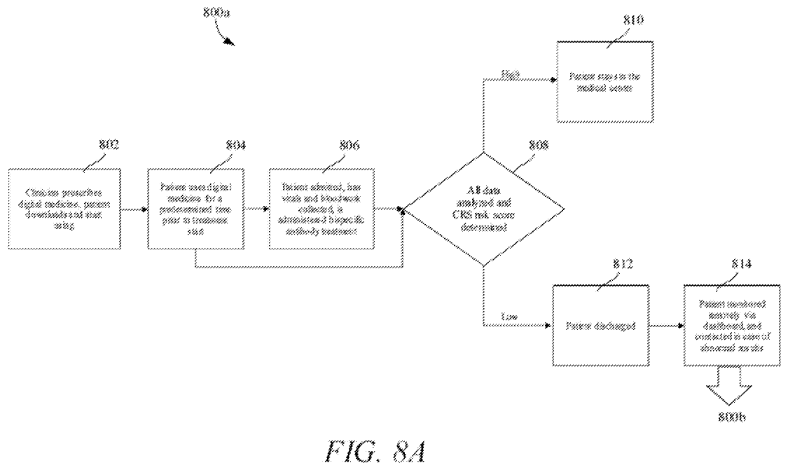

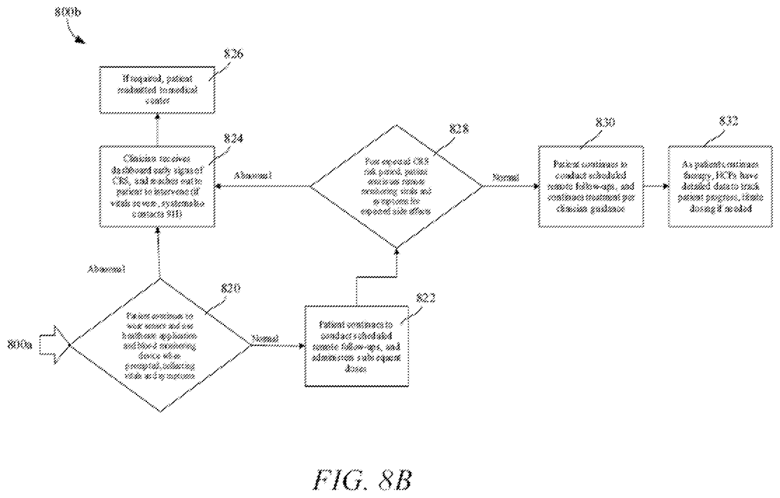

FIG. 8A shows a process flow 800a of CRS prediction for bispecific antibody treatment, according to several embodiments of this disclosure. The steps of the process flow 800a may be performed by any component of the operating environment 300 shown in FIG. 3. It should be understood that the steps of the process flow 800a are just examples and process flows with additional, alternative, and fewer number of steps should also be considered within the scope of this disclosure.

The process flow 800a may begin at step 802 when a clinician prescribes a digital medicine, which the patient may download and start using. The digital medicine may include, wearable devices and/or application for wearable devices, application for mobile devices, etc. At step 804, the patient may use the digital medicine for a predetermined time prior to the start of the bispecific antibody treatment. The predetermined time may include, for example, a few days, several hours, and/or any other type of time frame. At step 806, the patient may be admitted at a medical center (e.g., a hospital), has vitals and bloodwork collected, and is administered bispecific treatment. At step 808, the healthcare data is analyzed to determined CRS risk score (e.g., by using a trained machine learning model). In some embodiments, the risk score is determined without the administrating the treatment to the patient (e.g., step 806 may be skipped in some cases). If the CRS risk score is high, the patient may stay at the medical center for the treatment, as shown in step 810. However, if the CRS risk is low, the patient may be discharged at step 812, and monitored remotely via a dashboard (e.g., clinician dashboard) and contacted in case of abnormal results at step 814. The at-home monitoring process flow 800b is shown in FIG. 8B.

FIG. 8B shows the process flow 800b of at-home monitoring of a patient receiving a bispecific antibody treatment, according to several embodiments of this disclosure. The steps of the process flow 800b may be performed by any component of the operating environment 300 shown in FIG. 3. It should be understood that the steps of the process flow 800b are just examples and process flows with additional, alternative, and fewer number of steps should also be considered within the scope of this disclosure.

The process flow 800b may begin at step 820 (as a continuation from process flow 800a) where a patient may continue to wear sensor and use healthcare application and blood monitoring, when prompted. This continuous usage may continuously collect vitals and symptoms. If the continuously collected data indicate a normal progression (e.g., using a trained baseline model), the patient in step 822 may continue to schedule remote follow ups and subsequent treatment dosages may also be administered. However, if an abnormal progression is detected, a clinician at step 824 may receive a dashboard notification indicating early signs of CRS and reaches out to the patient for intervention. If required, the patient may be admitted to medical center at step 826.

The step after step 822 may be step 828 where the patient may continue remote monitoring vitals and expected side effects in the post expected CRS risk period. The process flow 800b may revert back to steps 824 and 826 if abnormal conditions are detected in step 828. However, if the conditions are detected to be normal in step 826, the patient in step 830 may continue to conduct scheduled remote follow-up and continue treatment based on clinician guidance. At step 832, as the patient continues therapy, clinicians have detailed data to track patient progress and titrate dosing as needed.

It will be appreciated by those skilled in the art that the present disclosure can be embodied in other specific forms without departing from the spirit or essential characteristics thereof. The presently disclosed embodiments are therefore considered in all respects to be illustrative and not restricted. The scope of the disclosure is indicated by the appended claims rather than the foregoing description and all changes that come within the meaning and range and equivalence thereof are intended to be embraced therein.

It should be noted that the terms “including” and “comprising” should be interpreted as meaning “including, but not limited to”. If not already set forth explicitly in the claims, the term “a” should be interpreted as “at least one” and “the”, “said”, etc. should be interpreted as “the at least one”, “said at least one”, etc. Furthermore, it is the Applicant's intent that only claims that include the express language “means for” or “step for” be interpreted under 35 U.S.C. 112 (f). Claims that do not expressly include the phrase “means for” or “step for” are not to be interpreted under 35 U.S.C. 112 (f).

1. A computer-implemented method comprising:
retrieving, prior to a treatment, first health data entered by a patient on a prescribed application being executed by a patient computing device;
retrieving, prior to the treatment, second health data passively collected by a prescribed wearable device;
deploying a machine learning model on the first health data and the second health data to determine whether the patient will likely experience cytokine release syndrome after the treatment; and
in response to the determination that the patient will likely experience the cytokine release syndrome, triggering a notification on a clinician dashboard.

2. The computer-implemented method of claim 1, further comprising:
training the machine learning model using a supervised approach by passing through labeled data of a cohort of patients through the model.

3. The computer-implemented method of claim 1, wherein the machine learning model comprises at least one of a regression model, a gradient boosted regression model, a logistic regression model, a random forest regression model, an ensemble model, a classification model, a deep learning neural network, a recurrent neural network for deep learning, or a convolutional neural network for deep learning.

4. The computer-implemented method of claim 1, wherein the first and the second health data is periodically received for a predetermined period of time prior to the treatment.

5. The computer-implemented method of claim 1, wherein the determination that the patient will likely experience the cytokine release syndrome is for a predetermined period of time after receiving the treatment.

6. The method of claim 1, wherein the first data comprises at least one of medical history, laboratory results, or patient profile.

7. The method of claim 1, wherein the second data comprises at least one of temperature, heart rate, blood pressure, or oxygen saturation.

8. The method of claim 1, wherein triggering the notification on the clinician dashboard comprises providing the notification to an electronic health record (EHR) system.

9. A computer-implemented method comprising:
retrieving, after a treatment, a first health data entered by a patient on a prescribed application being executed by a patient computing device;
retrieving, after the treatment, a second health data passively collected by a prescribed wearable device;
deploying a machine learning model on the first health data and the second health data to determine whether the patient will likely experience an adverse health condition; and
in response to the determination that the patient will likely experience the adverse health condition, triggering a notification on a clinician dashboard.

10. The computer implemented method of claim 9, further comprising:
training the machine learning model using a supervised approach by passing through labeled data of a cohort of patients through the model.

11. The computer-implemented method of claim 9, wherein the adverse health condition comprises at least one of cytokine release syndrome, infection, neurotoxicity, or cytopenia.

12. The computer implemented method of claim 9, wherein the machine learning model comprises at least one of a regression model, a gradient boosted regression model, a logistic regression model, a random forest regression model, an ensemble model, a classification model, a deep learning neural network, a recurrent neural network for deep learning, or a convolutional neural network for deep learning.

13. The computer-implemented method of claim 9, further comprising:
retrieving, after the treatment, bloodwork data for the patient; and
deploying the machine learning model on the first health data, the second health data, and the bloodwork data to determine whether the patient will likely experience the adverse health condition;
in response to determining that the patient will likely experience the adverse health condition, triggering the one or more notifications.

14. The computer-implemented method of claim 9, wherein the notification on a clinician dashboard comprises at least one of an indication that the clinician should contact the patient, an indication that dosage of a prescription medication is to be adjusted, or indication that the patient should be admitted to the hospital.

15. Th computer-implemented method of claim 9, further comprising:
in response to determining that the patient will likely experience the adverse healthcare condition, triggering a second notification to the prescribed application.

16. The computer-implemented method of claim 15, wherein the second notification comprises at least one of an indication that the patient should contract the clinician, an indication that the patient should pick up medication at the pharmacy, or an indication that the patient should contact emergency services.

17. A system comprising:
one or more processors; and
a non-transitory storage medium storing computer program instructions that when executed by the one or more processors cause the system to perform operations comprising:
retrieving a first health data entered by a patient, undergoing bispecific antibody treatment, on a prescribed application being executed by a patient computing device;
retrieving a second health data passively collected by a prescribed wearable device worn by the patient;
deploying a machine learning model on the first health data and the second health data to determine whether the patient will likely experience an adverse health condition; and
in response to the determination that the patient will likely experience the adverse health condition, triggering one or more notifications.

18. The system of claim 17, wherein the operations further comprise:
training the machine learning model using a supervised approach by passing through labeled data of a cohort of patients through the model.

19. The system of claim 17, wherein the machine learning model comprises at least one of a regression model, a gradient boosted regression model, a logistic regression model, a random forest regression model, an ensemble model, a classification model, a deep learning neural network, a recurrent neural network for deep learning, or a convolutional neural network for deep learning.

20. The system of claim 17, wherein the one or more notifications comprise at least one of a patient notification on the prescribed application and a clinician notification on a clinician dashboard.
